(19) United States
(12) Patent Application Publication
(10) Pub. No.: US 2005/0176956 A1
(43) Pub. Date: Aug. 11, 2005

(54) Process for the preparation of ganciclovir intermediate n2-acetyl-9-(1,3-diacetoxy-2-propoxymethyl) guanine

(76) Inventors: Jayachandra Suresh Babu, Gurgaon (IN); Purna Chandra Ray, New Delhi (IN); Chandra Has Khanduri, Gurgaon (IN); Yatendra Kumar, Gurgaon (IN)

Correspondence Address: RANBAXY INC., 600 COLLEGE ROAD EAST, SUITE 2100, PRINCETON, NJ, 08540, US

(21) Appl. No.: 10/492,338

(22) PCT Filed: Oct. 15, 2002
(86) PCT No.: PCT/IB02/04235

(30) Foreign Application Priority Data
Oct. 15, 2001 (IN) 1050/DEL/01

(51) Int. Cl. C07D 473/12

(52) U.S. Cl. 544/276

(57) ABSTRACT

The present invention relates to an improved process for the preparation of N²-acetyl-9-(1,3-diacetoxy-2-propoxymethyl)guanine, referred to here as the N-9 alkylated isomer, useful as intermediate for the preparation of antiviral compound, ganciclovir, including addition of a monoacetyl guanine, and optionally, addition of N²-acetyl-9-(1,3-diacetoxy-2-propoxymethyl)guanine, referred to herein as the N-7 alkylated isomer.

### FIELD OF THE INVENTION

The present invention relates to a process for the preparation of N²-acetyl-9-(1,3-diacetoxy-2-propoxymethyl)guanine, referred to here as N-9 alkylated isomer, useful as an intermediate for the preparation of the antiviral compound ganciclovir.

### BACKGROUND OF THE INVENTION

Ganciclovir, chemically known as 9-(1,3-dihydroxy-2-propoxymethyl)guanine has the structural formula I, 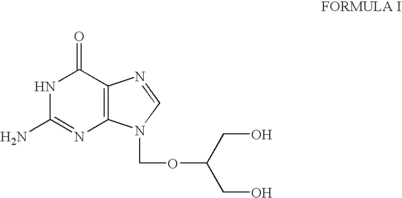
and is an important nucleoside having significant antiviral properties, being especially effective against viruses of the herpes family and a few other DNA viruses.

A number of methods are reported in the literature for the production of acylic purine nucleosides such as acyclovir and ganciclovir for example, methods which use guanine, diacetyl guanine, 2,6-dichloropurine, 2-amino-6-chloropurine (see U.S. Pat. No. 4,146,715 to Schaeffer); tetraacetylguanosine (J. Boryski et. al., Nucleosides and Nucleotides, 1989, 8, 529); acetylguanine (Japanese Patent Application No. 84-80685) or 2-chloro-6-iodopurine (J. R. Barrio et al., J. Med. Chem., 1980, 2, 572) as starting materials.

However, the simplest synthetic approach to the N-9 substituted guanine compounds involves the direct alkylation of appropriately substituted 2-aminopurines, for example, guanine derivatives. There are significant drawbacks to this approach as it typically results in a mixture of N-9 and N-7 (N²-acetyl-7-(1,3-diacetoxy-2-propoxymethyl)guanine)alkylation products. The undesirable N-7 isomers are produced in large amounts, which in turn necessitates tedious and inefficient methods of separation, such as silica gel chromatography, in order to obtain the desired N-9 isomer in acceptable purity. The use of halogenated purines is not a method of choice for industrial production, as these starting materials are expensive and difficult to acquire, and they require reaction with ammonia at high temperature and pressure in order to obtain guanine nucleosides, such as acyclovir, ganciclovir, and the like. Hence, it is highly desirable to develop a regioscpecific process for the manufacture of ganciclovir.

One such process which is regiospecific for the N-9 position was recently reported in U.S. Pat. No. 5,821,367 to Kumar et al. which includes reacting a protected guanine derivative with an alkylating agent selected from 2-oxa-1,4-butanediol diacetate; 1,4-diacetoxy-3-acetoxymethyl-2-oxabutane; 1,4-dibenzyloxy-3-acetoxymethyl-2-oxabutane in the absence of solvent or any acid catalyst to obtain the penultimate intermediates which are converted to acyclic nucleosides (acyclovir and ganciclovir). The conversion of N-7 isomer to N-9 isomer by heating a suspension of the N-7 isomer in an alkylating agent was reported in U.S. Pat. No. 6,043,364 to Kumar et al.

U.S. Pat. No. 5,583,225 to Chu et al. describes a process for the synthesis of purine nucleosides, particularly ganciclovir and acyclovir, where the deprotected guanine (diacetyl guanine) is reacted with the desired side chain in the presence of phosphoric or polyphosphoric acid at 120° C. Though the reaction time is short (3 hours) compared to prior processes, the product mixtures shows a low ratio of desired N-9 isomer in case of ganciclovir.

Each of the above methods has drawbacks in that the desired compounds are not obtained in particularly high yield and high purity, thus making the process complicated from the industrial point of view. Shortcomings in any of the parameters result in increased manufacturing cost, which impacts negatively on the desirability of the process.

It is therefore, desirable to solve the problems associated with the prior art and to provide an efficient process for the preparation and isolation of the desired N-9 isomer which improves the economics by resulting in higher yields of the desired isomer and less reaction time. The process should be easy to handle at commercial scale and avoid chromatographic separation of the N-9 and N-7 isomers and the increased cost associated with such a separation (cost of solvents, stationary phase).

### SUMMARY OF THE INVENTION

The invention provides an improved process for the production of N²-acetyl-9-(1,3-diacetoxy-2-propoxymethyl)guanine (referred to herein as the N-9 alkylated isomer), useful as an intermediate for the preparation of the antiviral compound ganciclovir. The invention results from the discovery that the presence of monoacetyl guanine (MAG) in the condensation reaction of diacetyl guanine with the side chain plays a crucial role in reaction completion.

Unless otherwise defined, all technical and scientific terms used herein have the same ordinary meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

Surprisingly, an efficient and selective process has been discovered for preparing ganciclovir. It has been discovered that the presence of monoacetyl guanine (MAG) in the condensation reaction of diacetyl guanine with the side chain plays a crucial role in reaction completion. Although MAG itself is not a suitable starting material for the alkylation, the addition of some quantity of MAG to the reaction mixture facilitates the reaction completion.

While not limiting the embodiments by reference to any theory of operation, a possible explanation for the better yields is that the reaction of monoacetyl guanine with the side chain produces acetic acid as a side product, which is believed to catalyze the reaction between the side chain and diacetyl guanine. This may be evidenced by the reaction of these intermediates in the presence of acetic acid. The alkylation reaction done in this mode gives better yield and quality of the desired N-9 isomer, which reduces the cost of the product, and reduces waste formation which is otherwise unavoidable.

Alkylation when performed below 100° C. was completed in 3045 hours and resulted in better yields of the desired N-9 isomer than when performed at a temperature above 100° C. The time required for alkylation at high temperature was less (6-10 hours) but the yields were less due to decomposition of the product of higher temperature.

Addition of the N-7 isomer during alkylation enhances the formation of N-9 isomer and suppresses the further formation of N-7 isomer. If the N-7 isomer has been obtained by recycling from a previous purification of an alkylation product mixture, the overall yield of the N-9 isomer is improved, and thus the overall yield of the desired product, that is, ganciclovir, is also improved.

The described process is simple and produces the N-9 isomer in greater than 95% purity, and which can directly be used for the preparation of ganciclovir. The process is cost effective and obviates chromatographic separation.

Thus the present process provides an efficient process for the preparation of ganciclovir which is convenient to operate on a commercial scale, reduces overall production costs, and gives the desired product in good yield and quality.

Accordingly, the present invention provides a process for the preparation of N-9 alkylated isomer of structural Formula V. 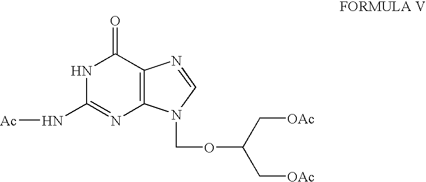
The process includes reacting diacetyl guanine of structural Formula 1 ml 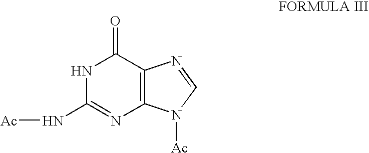
in the presence of monoacetyl guanine of structural Formula II 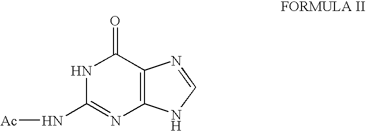
or acetic acid with 2-acetoxymethoxy-1,3-diacetoxy propane of structural Formula IV 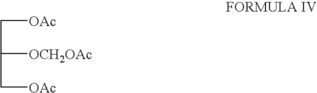
for 3-60 hours at a temperature of from about 50 to about 150° C. In some preferred embodiments, the reaction is carried out at a temperature of from about 50 to about 120° C., and in some more preferred embodiments, the reaction is carried out at a temperature of form about 50 to about 100° C.

The amount of monoacetyl guanine (MAG) of structural Formula II which is added to the alkylation reaction mixture can vary, and measured as the molar ratio of MAG:diacetyl guanine of structural formula III, can vary from about 0.05 to about 10.0. In some embodiments, the molar ratio can vary from about 0.05 to about 1.0, or from about 0.10 to about 0.50. In some further embodiments, the molar ratio of MAG:diacetyl guanine can vary from about 0.25 to about 0.50, for example, about 0.3 mole equivalent.

Additionally and optionally, acetic acid may also be added to the alkylation mixture, in an amount which can vary, and measured as the molar ratio of acetic acid:diacetyl guanine of structural formula III, can vary from about 0.01 to about 2.0, or from about 0.10 to about 1.0, or from about 0.40 to about 0.80, for example, about 0.59 or about 0.68.

Alkylation is desirably carried out in the presence of catalyst. Alkylation catalysts useful for the reaction are selected from sulfuric acid, methanesulfonic acid, p-toluene sulfonic acid and the like. In some embodiments, the preferred catalyst is p-toluene sulfonic acid.

The formation of the N-9 isomer can be enhanced by adding the N-7 isomer, having structural Formula VI, to the reaction mixture.

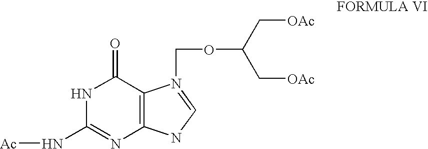

The amount of N-7 isomer which is added to the alkylation reaction mixture can vary, and measured as the molar ratio of N-7:diacetyl guanine of structural formula III, can vary from about 0.05 to about 10.0. In some embodiments, the molar ratio can vary from about 0.05 to about 1.0, or from about 0.10 to about 0.50. In some further embodiments, the molar ratio of N-7:diacetyl guanine can vary from about 0.25 to about 0.50.

The alkylation reaction can be performed in an inert organic solvent followed by a suitable work up and crystallization of N-9 and N-7 isomers from organic solvent or a mixture thereof Inert organic solventd for alkylation reaction are selected from benzene, toluene, xylene, acetonitrile, tetrahydrofuran, dimethylformamide, chloroform, dichloromethane, methyl iso-butyl ketone, and pyridine. In some preferred embodiments, the inert organic solvent is dimethylformamide.

N-9 and N-7 alkylated isomers of structural Formulae V and VI are separated from each other by methods known in the literature or by crystallization methods such as the following method.

Crystallization of a mixture including the N-7 and N-9 isomers will typically take place in an organic solvent, or a mixture of organic solvents. The choice of solvents is important for the separation of N-7 and N-9 isomers. The solvent system from which the isomers may be separated will desirably be selected from alcoholic solvents, which include lower alkanols, water-immiscible solvents, or a mixture thereof. The N-7 isomer will preferably be separated from the solvent system which has at least one lower alkanol. The lower alkanols include primary, secondary and tertiary alcohols having from one to six carbon atoms, for example, methanol, ethanol, n-propyl alcohol, isopropyl alcohol, isobutanol, n-butanol, tertiary butanol, or mixtures thereof. Most preferred being methanol, ethanol, or isopropyl alcohol. The N-9 isomer will preferably be separated from a solvent system which, in addition to alcoholic solvents, may contain water-immiscible solvents which include aromatic hydrocarbons such as benzene, toluene, or xylene, and chlorinated hydrocarbons such as chloroform, dichloromethane, or 1,2-dichloroethane.

After the N-7 isomer is separated, the concentration of the filtrate containing the N-9 isomer is adjusted by evaporation of the solvent or by dilution. The separation may comprise the last stage or stages of a reaction in which the mixture of N-7 and N-9 isomers is formed.

Methods known in the art may be used with the instant process to enhance any aspect of the process. For example, the solution containing the mixture of N-7 and N-9 isomers may be heated for dissolution, or it may be cooled to separate out the product or the slurry may further be cooled prior to filtration.

The N-9 isomer obtained after separation can be hydrolyzed to yield ganciclovir by methods known in the literature, including for example, J. E. Martin et. al., J. Med. Chem., (1983), 2, 759-761.

The invention will be further described in the following examples, which do not limit the scope of the invention described in the claims.

### EXAMPLES

### Example 1

### Preparation of N²-Acetyl-9-(1,3-diacetoxy-2-propoxymethyy)guanine

To a mixture of diacetylguanine (8 g, 34 mmol), monoacetylguanine (2 g, 10 mmole) and 2-acetoxymethoxy-1,3-diacetoxy propane (18 g, 72 mmole) in dimethylformamide (30 ml) was added p-toluene sulphonic acid (0.5 g, 2.6 mmole) at 90-100° C., and the reaction mixture was stirred for 35-45 hours. After reaction completion, solvent was distilled off and N-7 isomer (8.1 g) was isolated using methanol as crystallization solvent. Solvent was recovered to isolate the residue and the required N-9 isomer (7.62 g), was obtained by crystallization of the residue with toluene and methanol in 45% yield.

### Example 2

### Preparation of N²-Acetyl-9-(1,3-diacetoxy-2-propoxymethyy)guanine

To a mixture of diacetylguanine (24 g, 102 mmol), monoacetylguanine (6 g, 31 mmole) and 2-acetoxymethoxy-1,3-diacetoxy propane (54 g, 217 mmole) in dimethylformamide (90 ml) was added p-toluene sulphonic acid (1.5 g, 7.9 mmole) and N-7 isomer (15 g, 39 mmole) at 90-100° C. and the reaction mixture was stirred for 35-45 hours. After reaction completion, solvent was distilled off and N-7 isomer (12.5 g) was isolated using methanol as crystallization solvent. Solvent was recovered to get the residue and the required N-9 isomer (37 g) was obtained by crystallization of the residue from toluene and methanol in 73% yield.

### Example 3

### Preparation of N²-Acetyl-9-(1,3-diacetoxy-2-propoxymethyy)guanine

To a mixture of monoacetylguanine (20 g, 103 mmole) and 2-acetoxymethoxy-1,3-diacetoxypropane (36 g, 145 mmole) in dimethylformamide (60 ml) was added p-toluene sulphonic acid (1 g, 5.2 mmole) and stirred at 90-100° C. Reaction was not complete and even after 38 hours. The reaction mixture was filtered to remove unreacted monoacetylguanine (MAG) (3.85 g). Solvent was distilled off and N-7 isomer (4.65 g) was isolated using methanol as crystallization solvent. Solvent was recovered to get the residue and the required N-9 isomer (21.6 g) was obtained by crystallization of the residue from toluene and methanol. This N-9 isomer had 5.81% of MAG in it.

### Example 4

### Preparation of N²-Acetyl-9-(1,3-diacetoxy-2-propoxymethyy)guanine

To a mixture of diacetylguanine (20 g, 85 mmole), acetic acid (3.5 g, 58 mmole) and 1,3-diacetoxy-2-acetoxy-methoxy propane (36 g, 145 mmole) in dimethylformamide (60 ml) was added p-toluene sulphonic acid (1.0 g, 5 mmole) along with N-7 isomer (10 g) and the reaction mixture was stirred at 90-100° C. 35-45 hours.

After reaction completion, the solvent was distilled off and N-7 isomer (8.3 g) was isolated using methanol as crystallization solvent. Solvent was recovered to get the residue and the required N-9 isomer (20.1 g) was obtained by crystallization of the residue from toluene and methanol in 62% yield.

### Example 5

### Preparation of N²-Acetyl-9-(1,3-diacetoxy-2-propoxymethyy)guanine

To a mixture of diacetylguanine (9 g, 38 mmol), and 2-acetoxymethoxy-1,3-diacetoxypropane (18 g, 72.6 mmole) in dimethylformamide (27 ml) was added p-toluene sulphonic acid (1.35 g, 7.1 mmole) and N-7 isomer (4.5 g, 12 mmole) at 120-125° C. and the reaction mixture was stirred for 6-10 hours. After reaction completion, solvent was distilled off and N-7 isomer (3.5 g) was isolated using methanol as crystallization solvent. Solvent was recovered to get the residue and the required N-9 isomer (7.8 g,) was obtained by crystallization of the residue from toluene and methanol in 53.4% yield.

### Example 6

### Preparation of N²-Acetyl-9-(1,3-diacetoxy-2-propoxymethyy)guanine

To a mixture of diacetylguanine (10 g, 42.5 mmol), acetic acid (1.5 g, 25 mmole) and 2-acetoxymethoxy-1,3-diacetoxypropane (20 g, 80.6 mmole) in dimethylformamide (30 ml) was added p-toluene sulphonic acid (0.5 g, 2.6 mmole) and N-7 isomer (5 g, 13 mmole) at 120-125° C. and the reaction mixture was stirred for 6-10 hours. After reaction completion, solvent was distilled off and N-7 isomer (3.95) was isolated using methanol as crystallization solvent. Solvent was recovered to get the residue and the required N-9 isomer (8.5 g) was obtained by crystallization of the residue from toluene and methanol in 52.4% yield.

### Example 7

### Preparation of N²-Acetyl-9-(1,3-diacetoxy-2-propoxymethyy)guanine

To a mixture of diacetylguanine (8 g, 34 mmol), monoacetylguanine (2 g, 10 mmole) and 2-acetoxymethoxy-1,3-diacetoxypropane (20 g, 80 mmole) in dimethylformamide (30 ml) was added p-toluene sulphonic acid (0.5 g, 10 mmole) and N-7 isomer (5 g, 13 mmole) at 120-125° C. and the reaction mixture was stirred for 6-10 hours. After reaction completion, solvent was distilled off and N-7 isomer (4.1 g) was isolated using methanol as crystallization solvent. Solvent was recovered to get the residue and the required N-9 isomer (10.2 g) was obtained by crystallization of the residue from toluene and methanol in 60.8% yield.

### Example 8

### Preparation of N²-Acetyl-9-(1,3-diacetoxy-2-propoxymethyy)guanine

A mixture of diacetyl guanine (25 g, 0.106 mole), 2-acetoxymethoxy-1,3-diacetoxy propane (40.0 g, 0.161 mole), p-toluene sulfonic acid monohydrate (0.5 g) in N,N-dimethylformamide (75 ml) is heated at 95° C. to 100° C. under continuous stirring for 42 hours. After completion of the reaction, the solvents are removed under vacuum yielding a dark brown syrup. The syrup is dissolved by heating in methanol (60 ml). The resulting solution is stirred at room temperature, cooled to 0° C., stirred for 30 min. at 0-5° C. The crystallized material is filtered and washed with methanol (2×40 ml) to yield N²-acetyl-7-(1,3-diacetoxy-2-propoxymethyl)guanine (7.67 g).

The solvent from the filtrate is removed completely by distillation under reduced pressure to give an oily syrup. The oily syrup is dissolved in isopropyl alcohol and filtered through celite. The solvent is distilled off completely under vacuum. The residue is heated with a mixture of methanol (20 ml) and toluene (150 ml) at 60° C., stirred at room temperature and then at 0-5° C. for 30 minutes. The product is filtered and washed with a mixture of methanol and toluene (1:4) to yield N²-acetyl-9-(1,3-diacetoxy-2-propoxymethyl)guanine (11.0 g).

### Example 9

### Preparation of N²-Acetyl-9-(1,3-diacetoxy-2-propoxymethyy)guanine

A mixture of diacetyl guanine (10 g, 0.425 mole), 2-acetoxymethoxy-1,3-diacetoxy propane (150 ml, 0.605 mole), p-toluene sulfonic acid monohydrate (2.0 g), N²-acetyl-7-(1,3-diacetoxy-2-propoxymethyl)guanine (70 g) in N,N-dimethylformamide (400 ml) is heated at 90° C. to 100° C. under continuous stirring for 63 hours. After completion of the reaction, the solvents are removed under vacuum from the reaction mixture, yielding a dark brown syrup. The syrup is dissolved by heating in methanol (400 ml). The solution is cooled to 0° C., stirred for 1 hour at 0 to 5° C. The crystalline product is filtered and washed with methanol (2×1 00 ml) to yield N²-acetyl-7-(1,3-diacetoxy-2-propoxymethyl)guanine (69.0 g).

Solvent is removed completely from the filtrate and methanol (100 ml) and toluene (800 ml) are added to the residue and the mixture is heated to 60° C. and then cooled to 5° C. and stirred for 30 minutes. The crystalline product is filtered, washed with a mixture of methanol and toluene (1:4), dried at 60-65° C. to afford N²-acetyl-9-(1,3-diacetoxy-2-propoxymethyl)guanine (107.0 g).

### Example 10

### Preparation of N²-Acetyl-9-(1,3-diacetoxy-2-propoxymethyy)guanine

A mixture of diacetyl guanine (10 g, 0.425 mole), 2-acetoxymethoxy-1,3-diacetoxy propane (180 g, 0.725 mole), p-toluene sulfonic acid monohydrate (5.0 g), N²-acetyl-7-(1,3-diacetoxy-2-propoxymethyl)guanine (78 g) in N,N-dimethylformamide (350 ml) is heated at 95° C. to 100° C. under continuous stirring for 40 hours. After completion of the reaction, the solvents are removed under vacuum from the reaction mixture, yielding a dark brown syrup. The syrup is dissolved by heating in methanol (400 ml). The solution is cooled to 0° C., stirred for 1 hour at 0 to 5° C. The crystalline product is filtered and washed with methanol (50 ml) to yield N²-acetyl-7-(1,3-diacetoxy-2-propoxymethyl)guanine (54.1 g).

Solvent is removed completely from the filtrate and methanol (100 ml) and toluene (800 ml) were added to the residue and the mixture is heated to 60° C. and then cooled to 5° C. and stirred for 30 minutes. The crystalline product is filtered, washed with a mixture of methanol and toluene (1:4), dried at 60-65° C. to afford N²-acetyl-9-(1,3-diacetoxy-2-propoxymethyl)guanine (114.0 g).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A process for preparation of the N-9 alkylated isomer of structural formula V 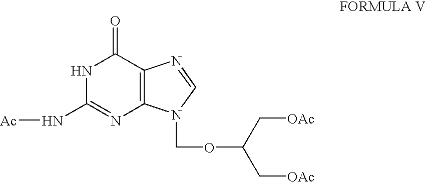
comprising alkylating diacetyl guanine of structural formula III 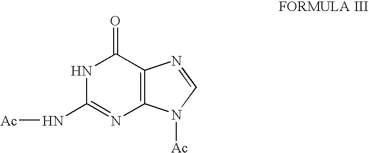
in the presence of monoacetyl guanine of structural Formula II 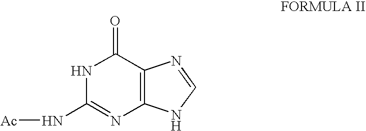
or acetic acid, with 2-acetoxymethoxy-1,3-diacetoxy propane of structural Formula IV 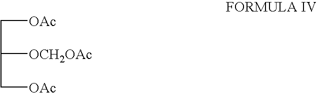
for 3-60 hours at a temperature from about 50 to 150° C.

2. The process of claim 1 wherein the alkylation is done in the presence of a catalyst selected from sulfuric acid, methanesulfonic acid or p-toluene sulfonic acid.

3. The process of claim 1, further comprising addition of the N-7 isomer of structural Formula VI 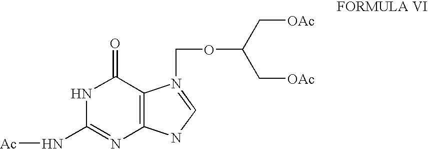
to the reaction mixture.

4. The process of claim 1 further comprises carrying out the reaction in an inert organic solvent selected from benzene, toluene, xylene, acetonitrile, tetrahydrofuran, dimethylformamide, chloroform, dichloromethane, methyl iso-butyl ketone or pyridine.

5. The process of claim 1 wherein between 0.1 mole equivalent to 0.5 mole equivalent of monoacetylguanine is used in reaction.

6. The process of claim 1 wherein 0.3 mole equivalent of monoacetylguanine is used in the reaction.

7. The process of claim 1 wherein between 0.4 mole equivalent to 0.8 mole equivalent of acetic acid is used in the reaction.

8. The process of claim 4 wherein 0.68 mole equivalent of acetic acid is used in the reaction.

9. The process of claim 1 further comprises the crystallization of N-9 and N-7 isomers from the reaction mixture after the alkylation is completed.

10. The process of claim 9 wherein N-9 isomer is separated from N-7 isomer by crystallizing in an organic solvent or a mixture thereof.

11. The process of claim 10 wherein the N-9 isomer is deprotected after the alkylation reaction.

12. A process for hydrolysis of the N-9 isomer of structural formula V 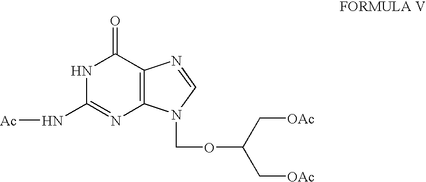
prepared by the process of claim 1 to give ganciclovir of Formula I

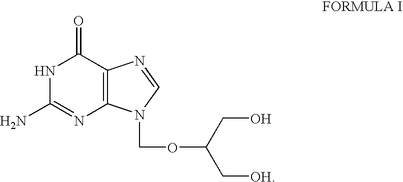

13. A process for preparation of the N-9 isomer of structural formula V 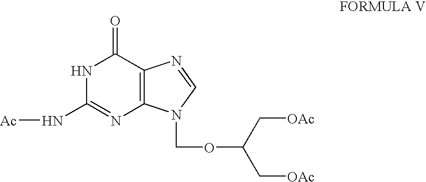
substantially described herein and exemplified by the examples.
